(19) United States
(12) Patent Application Publication
(10) Pub. No.: US 2023/0008996 A1
(43) Pub. Date: Jan. 12, 2023

(54) TOUCH INPUT DEVICE WITH DISINFECTION FUNCTION

(71) Applicant: Tzu-Hao LOU, Taipei City (TW)

(72) Inventor: Tzu-Hao LOU, Taipei City (TW)

(21) Appl. No.: 17/369,396

(22) Filed: Jul. 7, 2021

(51) Int. Cl. G06F 3/0354; A61L 2/26; A61L 2/10

(52) U.S. Cl. CPC G06F 3/03545; A61L 2/26; A61L 2/10; A61L 2202/122; A61L 2202/11

(57) ABSTRACT

A touch input device includes a container body internally defines a receiving chamber, a touch handle, and a disinfection member received in the receiving chamber for disinfecting the touch handle. The touch handle includes a handle portion and a touch portion, which are made of an electrical insulation material and an electrical conducting material, respectively. The handle portion has a part located in the receiving chamber to serve as an insertion section, and another part of the handle portion is exposed from the receiving chamber to serve as a grip section. The touch portion includes two connected touch sections, one of which is fitted on around the insertion section while the other one is connected to the grip section. When a user grips the touch handle to operate a touch electronic device, current in the user's body flows to the touch portion to increase the convenience in using the touch electronic device.

### FIELD OF THE INVENTION

The present invention relates to a touch input device for use with a touch electronic device, and more particularly, to a touch input device that not only has a disinfection function but also increases the touch sensitivity of a touch electronic device in response to the touch input device.

### BACKGROUND OF THE INVENTION

Following the constant progress in the technological field, many currently available touch electronic devices, such as tablet computer, smartphone and notebook computer, are provided with a touchscreen, allowing a user to directly touch the touchscreen and input instructions to control the touch electronic device. Currently, most of the users operate the touchscreen with their fingers of two hands or a stylus pen.

However, the stylus pen having been used to operate the touch electronic device over a long time would have tremendous bacteria attached thereto, and the user's hands carelessly contacting with the tip of the stylus pen would also be contaminated by the bacteria. In the event the user's eyes are touched by the bacteria-contaminated hands, the bacteria would invade into the user's body directly from the eyes to cause uncomfortable symptoms, such as cough, runny nose and fever.

### SUMMARY OF THE INVENTION

A primary object of the present invention is to provide an improved touch input device with disinfection function, which not only disinfects a touch handle of the touch input device in real time to always keep the touch handle in a disinfected state, but also largely increases the sensitivity of a touch electrical device, such as a touchscreen, a graphic board or a mobile device, in response to the touch handle.

To achieve the above and other objects, the touch input device with disinfection function according to a preferred embodiment of the present invention includes a container body, a touch handle and a disinfection member. The inside of the container body defines a receiving chamber for receiving the disinfection member.

The touch handle is selectively removably inserted into the receiving chamber to be disinfected by the disinfection member. The touch handle includes a handle portion and a touch portion. The handle portion is made of an electrical insulation material and includes an insertion section for inserting into the receiving chamber and a grip section exposed from the receiving chamber. The touch portion is made of an electrically conducting material and includes a first touch section for fitting on around the insertion section and a second touch section extended from the first touch section; and the second touch section is connected to the grip section.

In the preferred embodiment, the handle portion includes a recessed receiving slot formed on the insertion section and the grip section. The second touch section is received in the recessed receiving slot and the first touch section has a part received in the receiving chamber.

The present invention is characterized in that the touch input device includes a disinfection member to disinfect the touch handle of the touch input device at any time, so that the touch handle always maintains in a disinfected state. Further, in the present invention, the touch portion of the touch handle is connected not only to the insertion section of the handle portion, but also to the grip section of the handle portion, such that a user's hand gripping the touch handle to operate a touch electronic device is in contact with the touch portion, allowing current in the user's body to flow from the touch portion to the touch electronic device, such as a touchscreen, a graphic board or a mobile device. In this manner, the touch electronic device sensitivity in response to the touch handle can be largely increased to accordingly, increase the convenience in operating the touch electronic device.

### BRIEF DESCRIPTION OF THE DRAWINGS

The structure and the technical means adopted by the present invention to achieve the above and other objects can be best understood by referring to the following detailed description of the preferred embodiments and the accompanying drawings, wherein

FIG. 1 is an assembled perspective view of a touch input device according to a first preferred embodiment of the present invention;

FIG. 2 is an exploded and partially cutaway view of the touch input device of FIG. 1;

FIG. 3 is a cutaway view of a container body included in the touch input device according to the first preferred embodiment of the present invention;

FIG. 4 is an exploded front and partially sectioned view of the touch input device according to the first preferred embodiment of the present invention;

FIG. 5 is a block diagram showing the connection relation between an on/off switch, a processor, a disinfection member and a power supply unit included in the touch input device according to the first preferred embodiment of the present invention;

FIG. 6 is an exploded and partially phantom view of a touch handle included in the touch input device according to the first preferred embodiment of the present invention;

FIG. 7 is an assembled sectional view of the touch handle of FIG. 6;

FIG. 8A is a sectional view showing the use of the touch handle of FIG. 6 to operate a touch electronic device;

FIG. 8B is a sectional view of the touch input device of the present invention showing the touch handle is being inserted into a disinfection space in the disinfection member of the container body;

FIG. 8C is a sectional view of the touch input device of the present invention showing a first locating ring thereof is pressed against a second locating ring thereof;

FIG. 8D is a sectional view of the touch input device of the present invention showing the touch handle is in contact with the container body; and

FIG. 9 is a sectional view of a touch input device according to a second preferred embodiment of the present invention.

### DETAILED DESCRIPTION OF THE PREFERRED EMBODIMENTS

The present invention will now be described with some preferred embodiments thereof and by referring to the accompanying drawings. For the purpose of easy to understand, elements that are the same in the preferred embodiments are denoted by the same reference numerals.

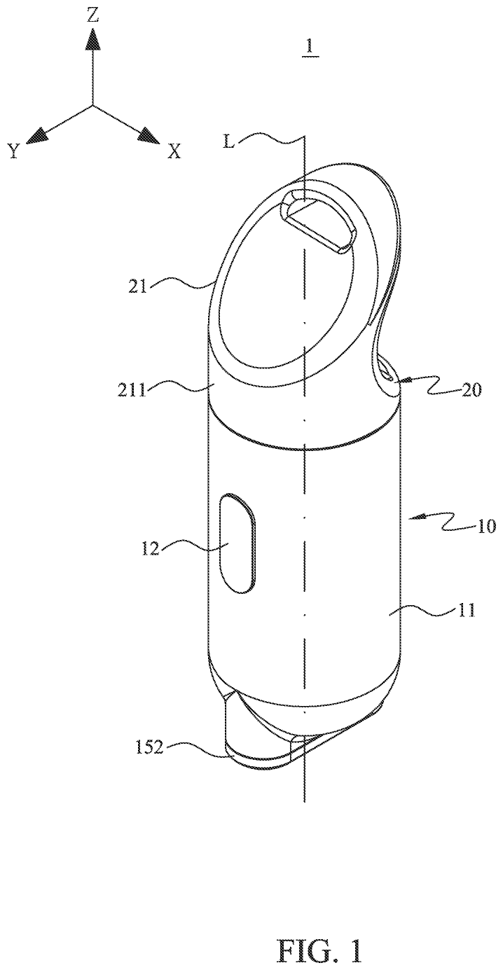

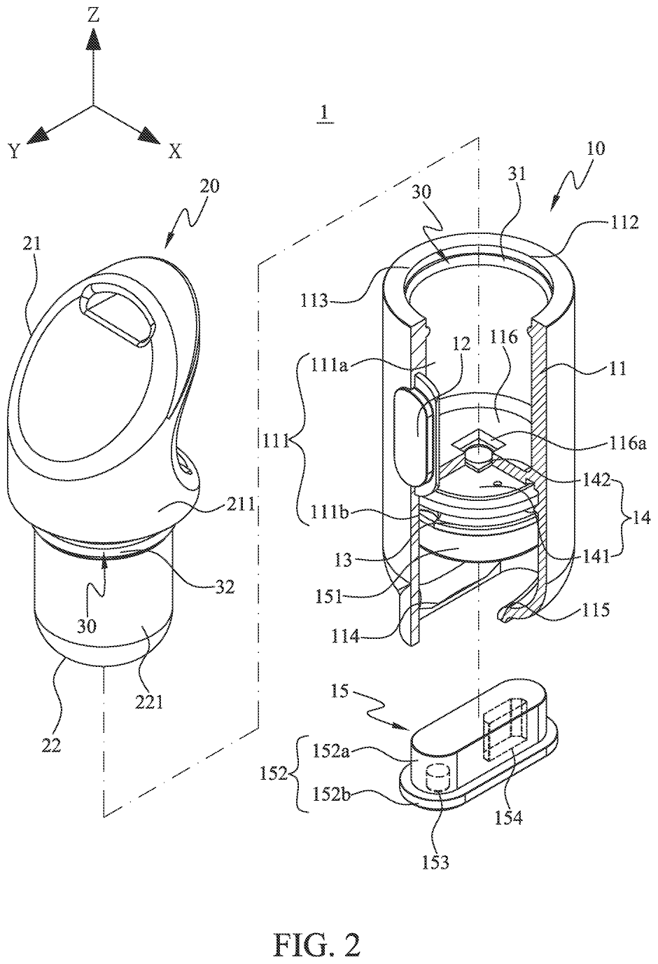

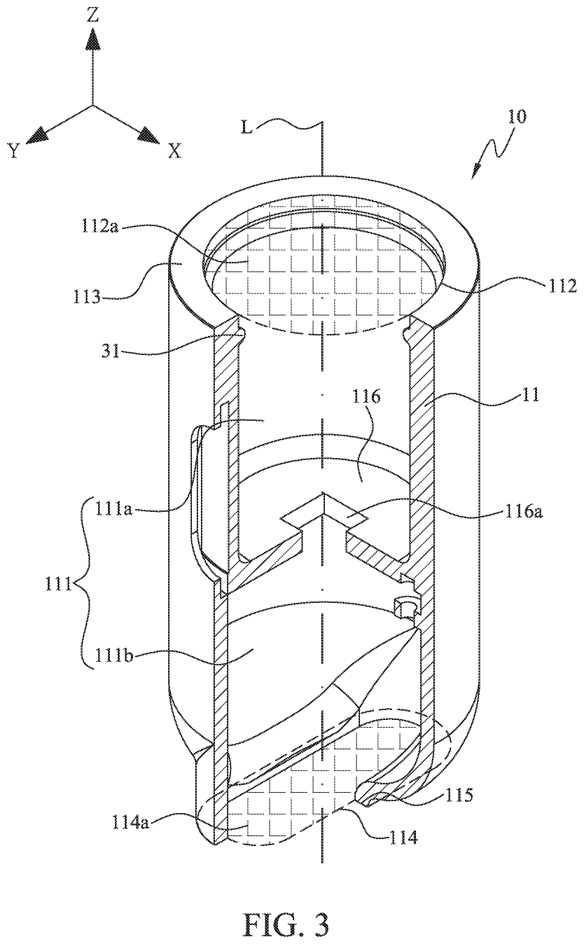

Please refer to FIGS. 1 and 2. A touch input device 1 according to a first preferred embodiment of the present invention includes a disinfection mechanism 10, a touch handle 20 and a locating member 30. As shown, the disinfection mechanism 10 includes a container body 11 that has a generally cylindrical configuration and extends along a longitudinal axis L parallel to the Z-axis. The container body 11 extends along the longitudinal axis L from an upper end of the container body 11 to a lower end of the container body 11 to internally define a receiving chamber 111. Please refer to FIG. 3. The receiving chamber 111 is a generally cylindrical chamber. More specifically, the container body 11 is provided at its upper end with an insertion opening 112 communicable with the receiving chamber 111 and an upper end contact surface 113 annularly extended around the insertion opening 112; and the container body 11 is further provided at its lower end with an mounting opening 114 communicable with the receiving chamber 111 and a lower end contact surface 115 annularly extended around the mounting opening 114.

In the first preferred embodiment, the insertion opening 112 is configured as a round opening and defines an inlet cross section 112a that is located substantially perpendicular to the longitudinal axis L, so that the insertion opening 112 is oriented in the same direction as the longitudinal axis L. Similarly, the mounting opening 114 defines a mounting cross section 114a that is substantially parallel to the inlet cross section 112a; and the mounting opening 114 is oriented to a direction away from the insertion opening 112. That is, the insertion opening 112 and the mounting opening 113 are oriented to two opposite directions.

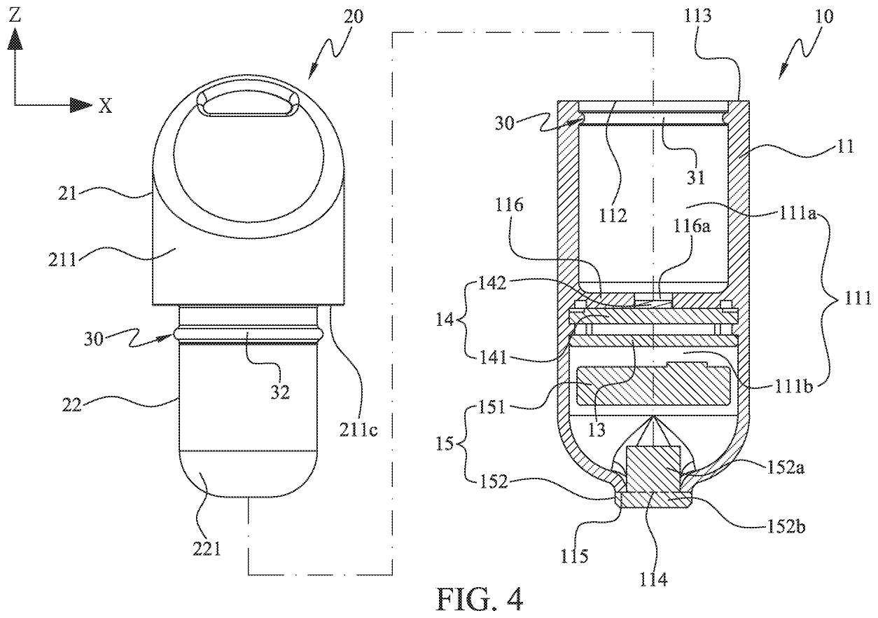

Please refer to FIGS. 3 and 4. In the receiving chamber 111 of the container body 11, a partition 116 is provided between the insertion opening 112 and the mounting opening 114. The partition 116 is located substantially parallel to the inlet cross section 112a of the insertion opening 112 and the mounting cross section 114a of the mounting opening 114, so that the partition 116 is generally perpendicular to the longitudinal axis L. The receiving chamber 111 is divided by the partition 116 into a disinfection space 111a communicable with the insertion opening 112 and a mounting space 111b communicable with the mounting opening 114. As can be seen in FIGS. 3 and 4, the disinfection space 111a is located above the partition 116, while the mounting space 111b is located below the partition 116, such that the disinfection space 111a, the partition 116 and the mounting space 111b are sequentially axially arranged from top to bottom along the longitudinal axis L. Further, the partition 116 is formed at a central area with a through hole 116a, so that the disinfection space 111a and the mounting space 111b are communicable with each other via the through hole 116a.

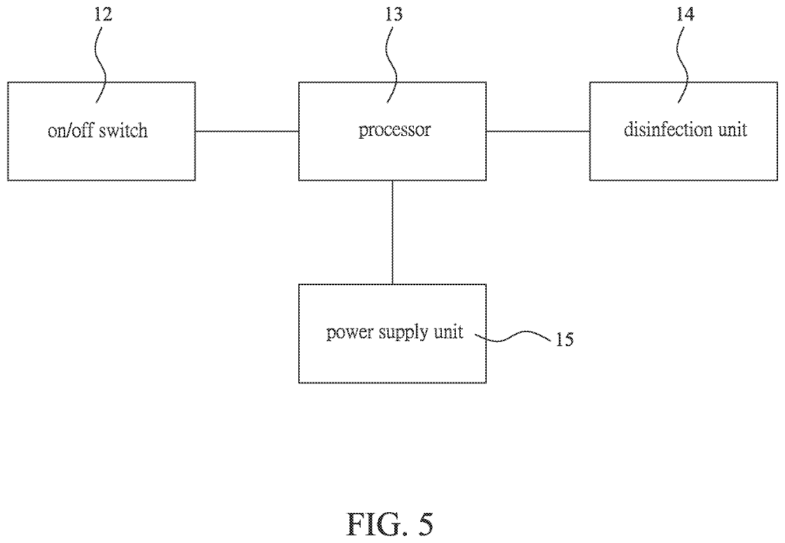

Please refer to FIGS. 2, 4 and 5. The disinfection mechanism 10 includes an on/off switch 12, a processor 13, a disinfection member 14 and a power supply unit 15 that are mounted in the container body 11. As shown, the on/off switch 12 is partially exposed from the container body 11 and is electrically connected to the processor 13. The processor 13 is fixed in the mounting space 111b of the container body 11 and is spaced from the partition 116 of the container body 11. The disinfection member 14 includes a light source circuit board 141 electrically connected to the processor 13 and a disinfection light source 142 electrically connected to the light source circuit board 141. The light source circuit board 141 is located between the partition 116 and the processor 113 and is accordingly, located in the mounting space 111b. That is, the partition 116, the light source circuit board 141 and the processor 13 are sequentially axially arranged along the longitudinal axis L. The disinfection light source 142 is located in the through hole 116a of the partition 116, so as to emit ultraviolet (UV) light toward the disinfection space 111a of the container body 11. As shown, a large part of the power supply unit 15 is located in the mounting space 111b. More specifically, the power supply unit 15 includes a power storage module 151 capable of storing electrical energy and a power charger 152 connected to the power storage module 151. The power storage module 151 is located below the processor 13, so that the processor 13 is located between the light source circuit board 141 and the power storage module 151. The power charger 152 is located below the power storage module 151, so that the power storage module 151 and the power charger 152 are sequentially axially arranged along the longitudinal axis L. The power charger 152 has a part that is extended into the mounting space 111b and is configured as a first connecting section 152a having a profile the same as that of the mounting cross section 114a. The other part of the power charger 152 is outward extended beyond the mounting opening 114 and is configured as a second connecting section 152b having a profile larger than that of the first connecting section 152a. Further, the second connecting section 152b is in contact with the lower end contact surface 115 of the container body 11, such that the power charger 152 is mounted at the mounting opening 114. An indicator light source 153 and a charging slot 154 are provided at the second connecting section 152b. The indicator light source 153 indicates the power storage module 151 is in the process of being charged. Both of the indicator light source 153 and the charging slot 154 are electrically connected to the power storage module 151.

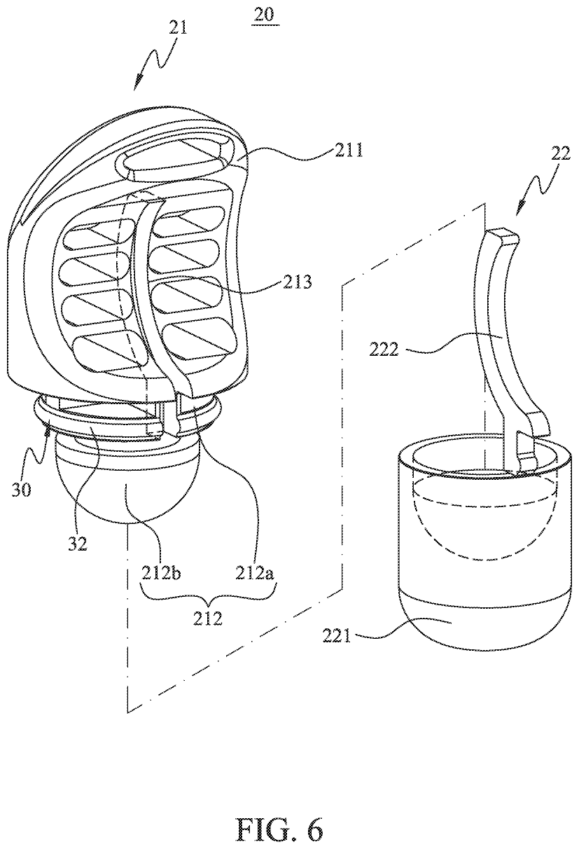

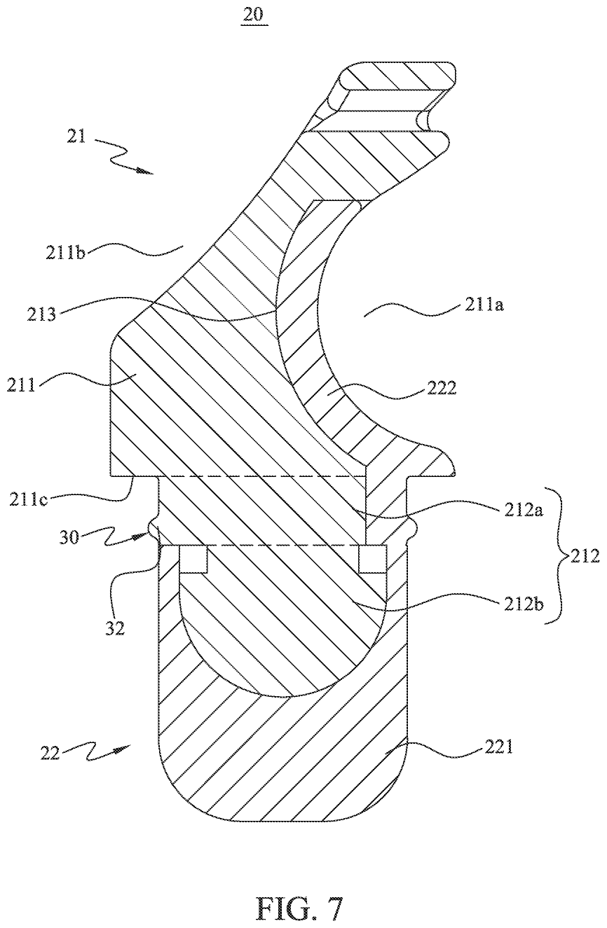

Please refer to FIGS. 2, 6 and 7. The touch handle 20 includes a handle portion 21 made of an electrical insulation material and a touch portion 22. The handle portion 21 has a part outward exposed from the insertion opening 112 of the container body 11 to serve as a grip section 211 located outside the disinfection space 111a. The other part of the handle portion 21 can be extended into the disinfection space 111a of the container body 11 via the insertion opening 112 to serve as an inserting section 212 extended from the grip section 211. As shown, the grip section 211 has a profile larger than that of the inlet cross section 112a of the insertion opening 112. Further, the grip section 211 is formed on one side with first recess 211a and on another opposite side with a second recess 211b. The second recess 211b has a shape different from that of the first recess 211a, such that the grip section 211 has a variable cross sectional area that gradually reduces along the longitudinal axis L in a direction away from the container body 11, allowing a user to conveniently hold the handle portion 21 at the grip section 211. Further, one side of the grip section 211 facing toward the container body 11 is a grip contact surface 211c annularly extended around the inserting section 212; meanwhile, the inserting section 212 includes a first inserting sub-section 212a that is located closer to the grip contact surface 211c and a second inserting sub-section 212b that has a profile smaller than that of the first inserting sub-section 212a. As shown, the handle portion 21 also includes a recessed receiving slot 213, a part of which is formed on the first recess 211a of the grip section 211 and the remaining part of which is formed on the first inserting sub-section 212a of the inserting section 212.

As shown, the touch portion 22 of the touch handle 20 is made of an electrically conducting material and includes a first touch section 221 and a second touch section 222 extended from the first touch section 221. The first touch section 221 is fitted on around the second sub-section 212b of the inserting section 212, such that the entire second inserting sub-section 212b is located in the first touch section 221. When the first touch section 221 is fitted on around the second inserting sub-section 212b, an outer periphery of the first touch section 221 is flush with the first inserting sub-section 212a of the inserting section 212. The second touch section 222 has a shape different from that of the first touch section 221 and is configured for setting in the recessed receiving slot 213 of the handle portion 21, such that the second touch section 222 is simultaneously snap-fitted in the grip section 211 and the inserting section 212. Further, when the inserting section 212 of the handle portion 21 is located in the disinfection space 111a of the container body 11, the grip contact surface 211c of the handle portion 21 is abutted on the upper end contact surface 113 of the container body 11 while a part of the second touch section 222 is locate in the disinfection space 111a.

Please refer to FIG. 4 again. The locating member 30 is provided between the container body 11 and the touch handle 20 to prevent the inserting section 212 of the handle portion 21 from moving out of the disinfection space 111a of the container body 11. As shown, the locating member 30 includes a first locating ring 31 formed on the container body 11 and a second locating ring 32 formed by radially outward extending from the inserting section 212. The first locating ring 31 is located in the disinfection space 111a and has an inner periphery larger than an outer periphery of the second locating ring 32. In the preferred embodiment, the first locating ring 31 does not deform when it is subjected to an externally applied compression, while the second locating ring 32 will deform when it is subjected to an externally applied compression.

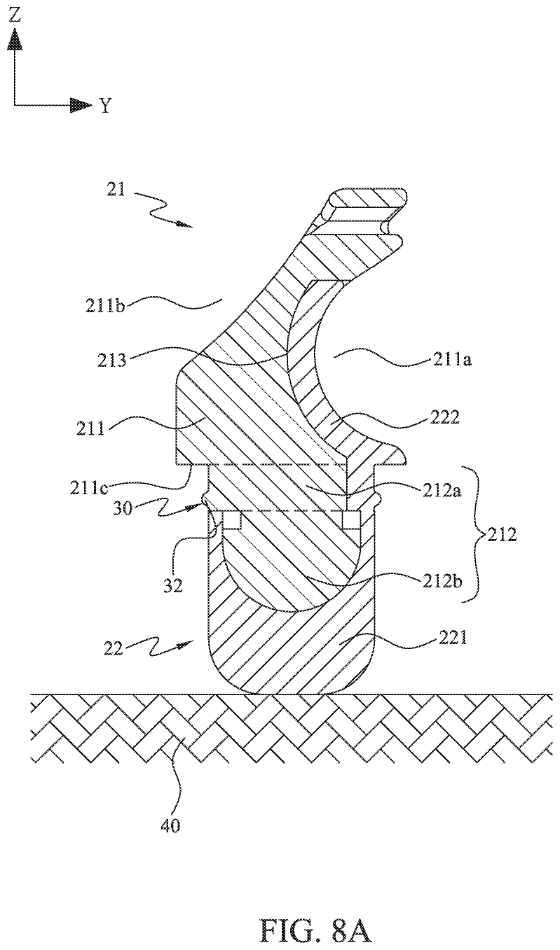

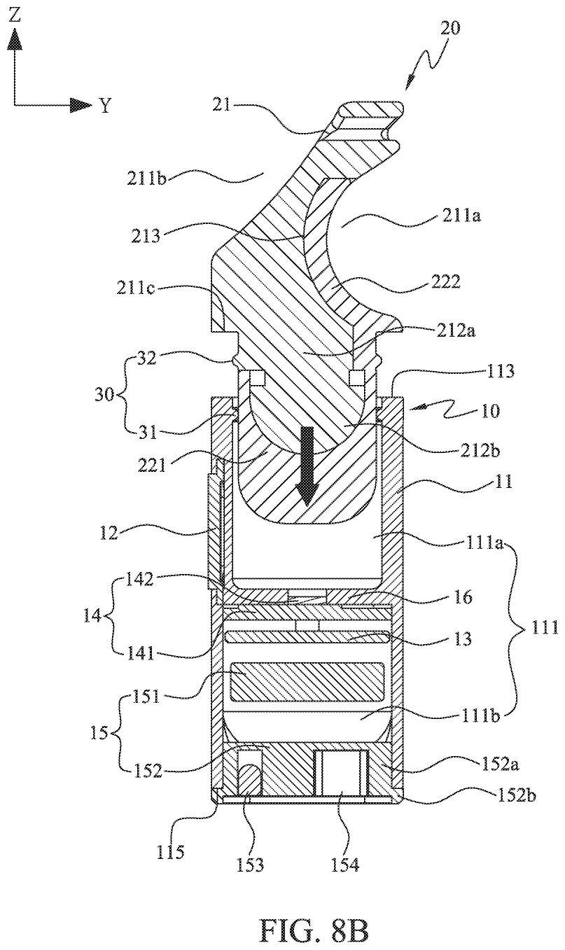

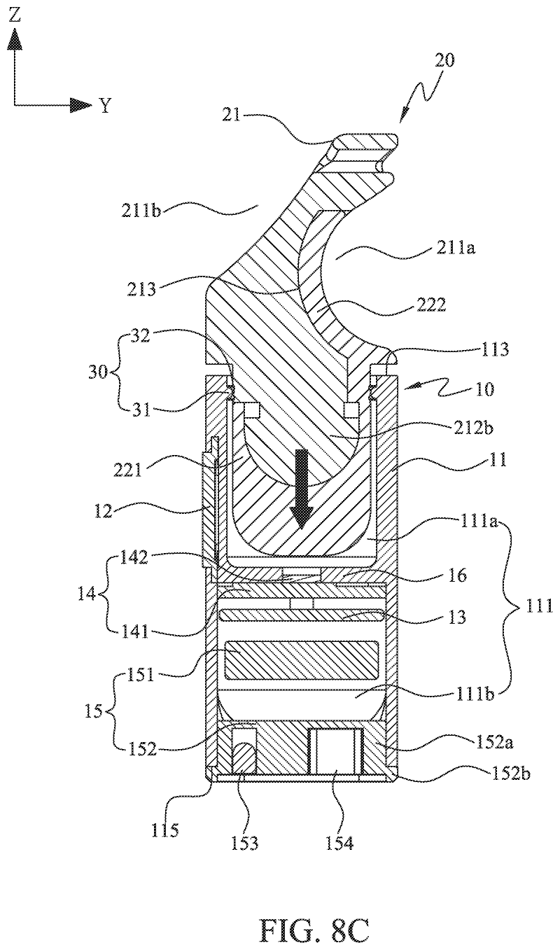

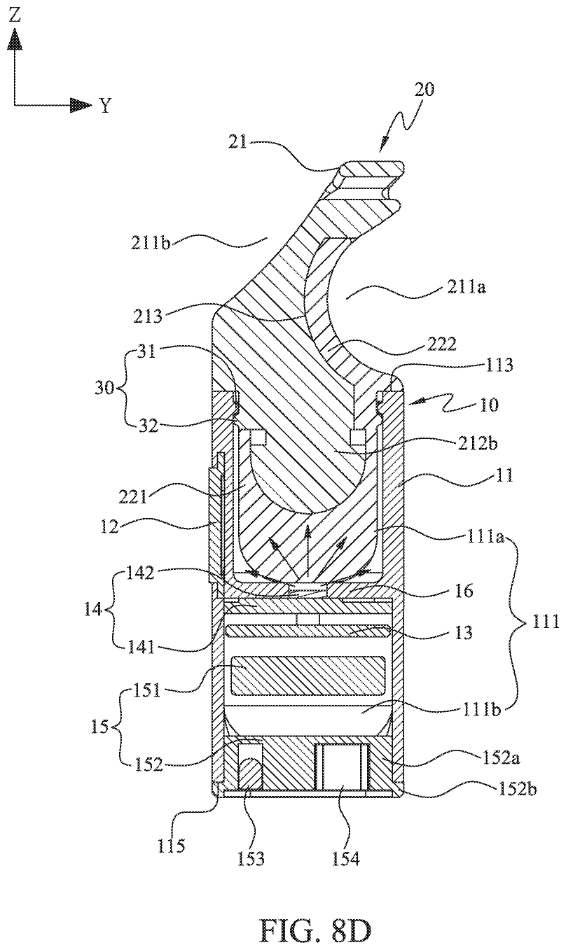

Please refer to FIG. 8A. To use the touch input device 1 according to the present invention, the user grips the touch handle 20 at the grip section 211 with the user's finger getting in contact with the second touch section 222 of the touch portion 22. so that current in the user's body flows from the second touch section 222 to the first touch section 221 of the touch portion 22. With these arrangements, when the touch handle 20 is used to operate a touch electronic device 40, such as a touchscreen, a graphic board or a mobile device, the touch electronic device sensitivity in response to the touch handle 20 is largely increased.

Please refer to FIG. 8B. When the touch handle 20 is not in use, the first touch section 221 of the touch handle 20 can be inserted through the insertion opening 112 of the container body 11 into the disinfection space 111a of the receiving chamber 111, so that the second locating ring 32 of the locating member 30 is brought to gradually approach the first locating ring 31 of the locating member 30. Since the profile of the insertion opening 112 is round and the first touch section 221 of the touch portion 22 has a cylindrical profile, the first touch section 221 can be easily inserted into the disinfection space 111a.

Please refer to FIGS. 8C and 8D. When the first locating ring 31 and the second locating ring 32 of the locating member 30 are located at substantially the same height position, the second locating ring 32 is compressed by the first locating ring 31 to have a deformed profile, which allows the outer periphery of the second locating ring 32 to match the inner periphery of the first locating ring 31. Thereafter, the first touch section 221 of the touch handle 20 is brought to gradually approach the partition 116 in the container body 11. Finally, the grip contact surface 211c of the touch handle 20 is in contact with the upper end contact surface 113 of the container body 11 and the second locating ring 32 is now moved to a height location lower than that of the first locating ring 31. At this point, the on/off switch 12 of the disinfection mechanism 10 can be depressed for the disinfection light source 142 of the disinfection member 14 to emit UV light toward the disinfection space 111a of the container body 11, and the UV light of the disinfection light source 142 disinfects the first touch section 221 to always keep the first touch section 221 in a disinfected state.

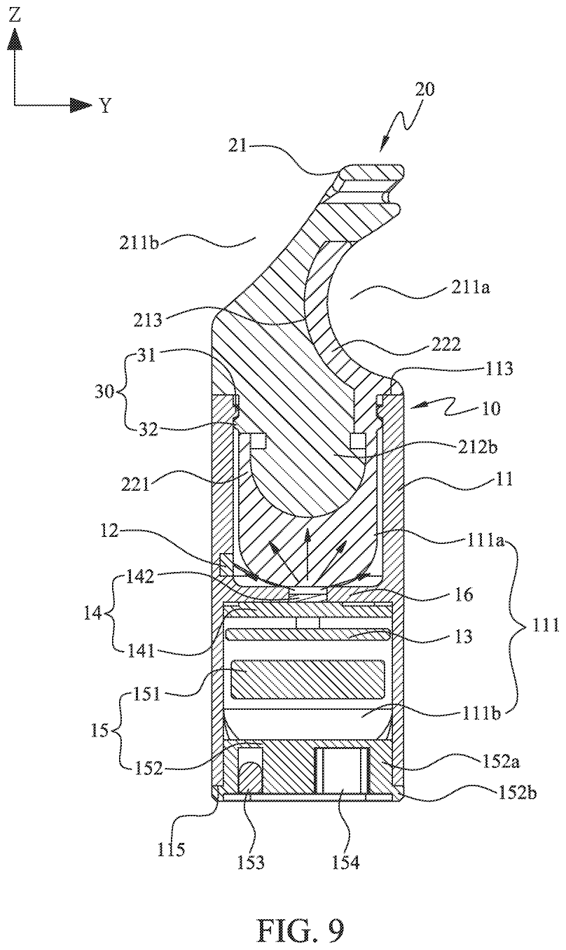

Please refer to FIG. 9, in which a touch input device according to a second preferred embodiment of the present invention is shown. The second preferred embodiment is different from the first preferred embodiment only in the mounting position of the on/off switch. Since all other parts of the second preferred embodiment are similar to those of the first preferred embodiment, they are not repeatedly described herein. As shown, in the second preferred embodiment, the on/off switch 12 is disposed inside the disinfection space 111a of the container body 11. When the first touch section 221 of the touch handle 20 is inserted into the disinfection space 111a of the receiving chamber 111 and the grip contact surface 211c of the touch handle 20 is in contact with the upper end contact surface 113 of the container body 11, the first touch section 221 will abut on the on/off switch 12 for the disinfection light source 142 of the disinfection member 14 to emit UV light toward the first touch section 221. In this manner, the first touch section 221 is always maintained in a disinfected state.

The present invention has been described with some preferred embodiments thereof and it is understood that many changes and modifications in the described embodiments can be carried out without departing from the scope and the spirit of the invention that is intended to be limited only by the appended claims.

What is claimed is:

1. A touch input device with disinfection function, comprising a container body, a touch handle and a disinfection member;, the inside of the container body defines a receiving chamber for receiving the disinfection member and the touch handle being selectively removably inserted into the receiving chamber to be disinfected by the disinfection member, being characterized in that:
the touch handle includes a handle portion and a touch portion; the handle portion being made of an electrical insulation material and including an insertion section for inserting into the receiving chamber and a grip section exposed from the receiving chamber; the touch portion being made of an electrically conducting material and including a first touch section for fitting on around the insertion section and a second touch section extended from the first touch section; and the second touch section being connected to the grip section.

2. The touch input device with disinfection function as claimed in claim 1, wherein the handle portion includes a recessed receiving slot formed on the insertion section and the grip section; and the second touch section being set in the recessed receiving slot and the second touch section having a part received in the receiving chamber.
